(19) United States
(12) Patent Application Publication
(10) Pub. No.: US 2009/0133696 A1
(43) Pub. Date: May 28, 2009

(54) VENTILATION STABILIZATION SYSTEM

(76) Inventors: John E. Remmers, Calgary (CA); John J. Laprairie, Calgary (CA)

Correspondence Address: CHRISTENSEN, O'CONNOR, JOHNSON, KINDNESS, PLLC, 1420 FIFTH AVENUE, SUITE 2800, SEATTLE, WA, 98101-2347, US

(21) Appl. No.: 12/258,286

(22) Filed: Oct. 24, 2008

Related U.S. Application Data
(60) Provisional application No. 60/983,149, filed on Oct. 26, 2007.

(51) Int. Cl. A61M 16/00

(52) U.S. Cl. 128/204.26

(57) ABSTRACT

There is an apparatus including a mandible positioner for positioning the mandible of a patient with respect to the maxilla of the patient and a breathing assistance apparatus. The breathing assistance apparatus has a sensor arranged to detect at least one element representative of a breathing state of the patient. A source of breathing gas includes a patient interface and has at least a first operable position and a second operable position. The source of breathing gas provides a different ratio of carbon dioxide and oxygen to the patient when in the first operable position than in the second operable position. The source of breathing gas may be moved between the first operable position and the second operable position in response to signals from the sensor. For example, the source of breathing gas may provide rebreathed air to the patient in the first position and atmospheric air in the second position. An apparatus for interfacing with a patient to allow breathing gas to be provided to the patient is also provided.

### BACKGROUND

Central sleep apnea is a type of sleep-disordered breathing that is characterized by a failure of the sleeping brain to generate regular, rhythmic bursts of neural activity. The resulting cessation of rhythmic breathing, referred to as apnea, represents a disorder of the respiratory control system responsible for regulating the rate and depth of breathing, i.e. overall pulmonary ventilation. Central sleep apnea should be contrasted with obstructive sleep apnea, where the proximate cause of apnea is obstruction of the pharyngeal airway despite ongoing rhythmic neural outflow to the respiratory muscles. The difference between central sleep apnea and obstructive sleep apnea is clearly established, and the two can co-exist.

Obstructive sleep apnea occurs when physical obstruction of the airway passage occurs, for example due to the pharynx flopping around. Nasal continuous positive airway pressure (CPAP) is the standard medical treatment for obstructive sleep apnea. Nasal CPAP involves the application of positive airway pressure to the nasal airway, thereby increasing the intrapharyngeal pressure and maintaining pharyngeal patency. A problematic aspect of this therapeutic approach is the establishment of an interface between the pressure generating device and the nasal airway. For this purpose, a number of nose masks have been devised and are commercially available. Another problematic feature of nasal CPAP therapy is the occurrence of the flow of gas from the pharynx through the mouth and into the atmosphere; that is, mouth leaks. This leakage of gas from the pharynx out the mouth causes an increase in flow of air through the nose and can lead to rhinitis. In addition, mouth leaks are disturbing for the patient and bed partner. Finally, certain applications of nasal CPAP require establishment of a leak-free interface and this implies an elimination of mouth leaks. Traditionally, the problem of mouth leaks has been addressed using a chin strap or a full face mask. Both present substantial difficulties for the patient and are often ineffective.

The elimination of mouth leaks during nasal CPAP therapy is challenging because the mouth consists of a fixed upper dental arch or maxillary arch, and a movable lower dental arch, or mandible. In addition, establishing a seal at the lips can be difficult. Thus, in order to establish a mouth seal, one needs to stabilize the mandible and establish a seal at the lips. One approach used to prevent mouth leaks is to use a full face mask covering both the nose and the mouth. However, a full face mask often fails to stabilize the mandible. Accordingly, when substantial forces are used to seal the full face mask against the skin over the lower lip and chin, the mandible is forced backwards. This effect of retruding the mandible may cause a backward movement of the tongue and narrowing of the pharynx. The difficulties of the full face mask are widely appreciated. Additionally, a full face mask is more likely to induce claustrophobia than a nose mask or a nose and mouth interface.

Central sleep apnea, in contrast to obstructive sleep apnea, relates more to defects in the breathing control systems. While central sleep apnea can occur in a number of clinical settings, it is most commonly observed in association with heart failure or cerebral vascular insufficiency. Cheyne Stokes breathing is a condition in which a person has increased breath volume (tidal volume) with each breath and increased frequency of breathing. This is a form of breathing instability and it may be caused by central sleep apnea. There are chemoreflex feedback loops that control breathing and Cheyne Stokes breathing results from increased gain in the feedback loops. One feedback loop is called the peripheral feedback loop and it involves a CO₂ and O₂ sensor in the carotid artery. If the gain in this loop is too high, it can cause breathing instability. Other causes of central sleep apnea and Cheyne Stokes breathing include circulatory delay and pharyngeal instability.

Both pharyngeal instability and increase gain of chemoreflex loops underlie the pathogenesis of central sleep apnea. While continuous positive airway pressure (CPAP) has been traditionally used to stabilize the pharynx, this can also be achieved with mandibular protrusion. In fact, central sleep apnea can be seen as an emergent phenomenon in the setting of mandibular protrusion treatment of obstructive sleep apnea. The mandibular protrusion device is used to adjust the position of the mandible relative to the maxilla. The applicant is not aware of mandibular protrusion devices being used for central sleep apnea treatments. Additionally, although mandibular protrusion devices are known in the art for the treatment of obstructive sleep apnea, they are not always effective.

The effects of an abnormally high gain in the chemoreflex feedback loops can be mitigated by controlled rebreathing. In this approach, a leak-free interface is applied and an external dead space is increased at critical times during the central sleep apnea cycle. Thus, transient rebreathing occurs during hyperventilatory phases in order to mitigate the increase in alveolar ventilation that occurs at these times.

Controlled re-breathing is known in the art for the treatment of central sleep apnea. It is described for example in U.S. Pat. No. 7,073,501, patented on Jul. 11, 2006, which is incorporated here by reference. In controlled re-breathing the patient re-breathes exhaled air, which has an increased CO₂ and reduced O₂ content. Controlled re-breathing affects the peripheral feedback loop and reduces loop gain. Controlled re-breathing is not always effective. In controlled re-breathing an interface is required to control re-breathing. The patient could be made to re-breathe all night through a permanently connected tube providing a dead space, but the patient will get a headache, may suffer other problems and the body will adapt to the continuous supply of excess CO₂.

### SUMMARY

In an embodiment there is provided an apparatus including a mandible positioner for positioning the mandible of a patient with respect to the maxilla of the patient and a breathing assistance apparatus. The patient has a breathing state. The breathing assistance apparatus has a sensor arranged to detect at least one element representative of the patient's breathing state. A source of breathing gas includes a patient interface and has at least a first operable position and a second operable position. The source of breathing gas provides a different ratio of carbon dioxide and oxygen to the patient when in the first operable position than in the second operable position. The source of breathing gas is movable between the first operable position and the second operable position in response to signals from the sensor.

Another embodiment concerns a method for assisting the breathing of a patient comprising the steps of protruding the mandible of a patient with a mandibular protrusion device. In this method, the breathing of a patient is detected and it is determined whether abnormal breathing conditions are present. An amount of carbon dioxide concentration provided to the patient is changed when abnormal breathing conditions are determined to be present.

Yet another embodiment concerns an apparatus for enhancing the breathing of a patient having a breathing state. The apparatus includes a mandibular positioning device and an interface. The interface is adapted to provide breathing gas to a breathing orifice of the patient. A sensor is attached to the interface. The sensor detects at least one element representative of the patient's breathing state. A fluid manifold connected to an exterior air source is connected to the interface. An exit is connected to the interface. A valve is operably connected to the sensor to vary the amount of flow of exhaled gases from the patient into the fluid manifold.

Still another embodiment concerns an apparatus for assisting the breathing of a patient having a breathing state. The apparatus includes a mandibular positioning device and an interface. The interface is adapted to provide breathing gas to a breathing orifice of the patient. A fluid manifold is connected to the interface. A sensor is attached to the fluid manifold. The sensor detects at least one element representative of the patient's breathing state. An exterior pressurized air source is connected to the interface. The exterior pressurized air source provides air with a higher content of oxygen gas than atmospheric air to the patient in response to signals from the sensor.

In another embodiment, an apparatus for interfacing with a patient to allow breathing gas to be provided to the patient is provided. The apparatus comprises a nasal interface having a nose seal, and an oral interface having a mouth seal, a mandibular positioning device for positioning the mandible of a patient with respect to a maxilla of the patient and a gas passage through at least one of the nasal interface and the oral interface through which breathing gas can be provided to the patient.

In another embodiment, an apparatus for interfacing with a patient to allow breathing gas to be provided to the patient is provided. The apparatus comprises an oral interface having a mouth seal comprising an internal flange for sealing around an interior of the mouth, and an external flange for sealing around an exterior of the mouth, and a mandibular positioning device for positioning the mandible of a patient with respect to a maxilla of the patient.

In another embodiment, an apparatus for interfacing with a patient to allow breathing gas to be provided to the patient is disclosed. The apparatus comprises a mandibular positioning device for positioning the mandible of a patient with respect to a maxilla of the patient. The apparatus further comprises one or more of the following sets of features: a nasal interface having a nose seal, an oral interface having a mouth seal, and a gas passage through at least one of the nasal interface and the oral interface through which breathing gas can be provided to the patient; and an oral interface having a mouth seal comprising an internal flange for sealing around an interior of the mouth, and an external flange for sealing around an exterior of the mouth.

These and other aspects of the device and method are set out in the claims, which are incorporated here by reference.

### BRIEF DESCRIPTION OF THE FIGURES

Embodiments will now be described with reference to the figures, in which like reference characters denote like elements, by way of example, and in which:

FIG. 1 is a side perspective view of an oral interface on a patient;

FIG. 2 is a front perspective view of the oral interface of FIG. 2 on a patient;

FIG. 3 is a partial front perspective view of a dental appliance in a patient's mouth;

FIG. 4 is a front perspective view of the dental appliance of FIG. 3;

FIG. 5 is a top perspective view of a dental appliance on an oral interface;

FIG. 6 is a side perspective view of the oral interface and dental appliance of FIG. 5;

FIG. 7 is a front perspective view of the oral interface and dental appliance of FIG. 5;

FIG. 8 is a side perspective view of an oral interface with headgear connected to a nasal interface with headgear on a patient;

FIG. 9 is a front perspective view of the oral interface and nasal interface of FIG. 8 on a patient;

FIG. 10 is a partial perspective view of a patient having a mandible protrusion device in combination with a breathing assistance apparatus;

FIG. 11 is a partial side perspective view of a second embodiment of a mandible protrusion device in combination with a second embodiment of a breathing assistance apparatus;

FIG. 12 is a side view of a mandibular protruder showing upper and lower portions of a dental appliance;

FIG. 13 is a side view of a second embodiment of a mandibular protruder;

FIG. 14 is a top view of another embodiment of a mandibular protruder;

FIG. 15 is a plan view of an embodiment of a controlled-rebreathing apparatus;

FIG. 16 is a plan view of an embodiment of an oral interface in a controlled-rebreathing system;

FIG. 17 is perspective view of an embodiment of an oral interface;

FIG. 18 is a side view of an embodiment of a passive loop gain modulation system; and

FIG. 19 is a side view of the passive loop gain modulation system of FIG. 18 with a flow meter and a computer.

### DETAILED DESCRIPTION

In the claims, the word “comprising” is used in its inclusive sense and does not exclude other elements being present. The indefinite article “a” before a claim feature does not exclude more than one of the feature being present. Each one of the individual features described here may be used in one or more embodiments and is not, by virtue only of being described here, to be construed as essential to all embodiments as defined by the claims.

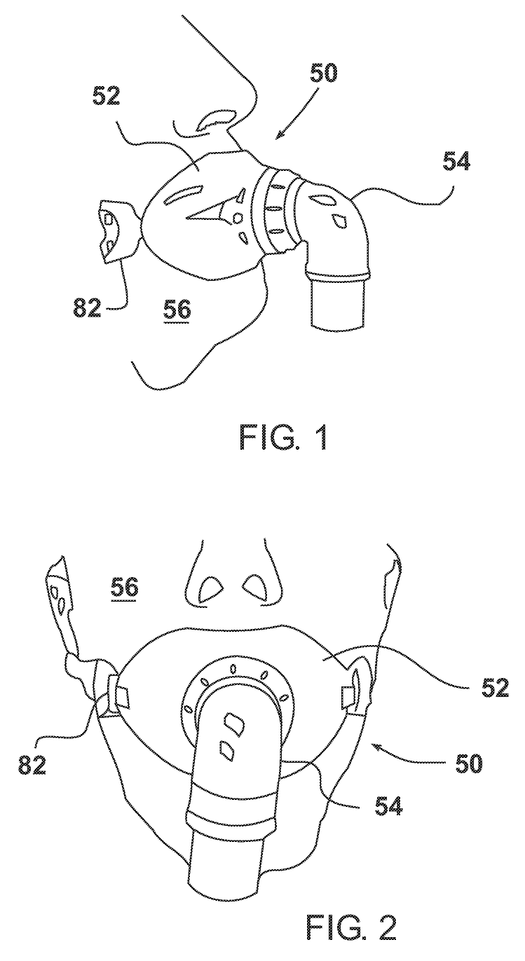

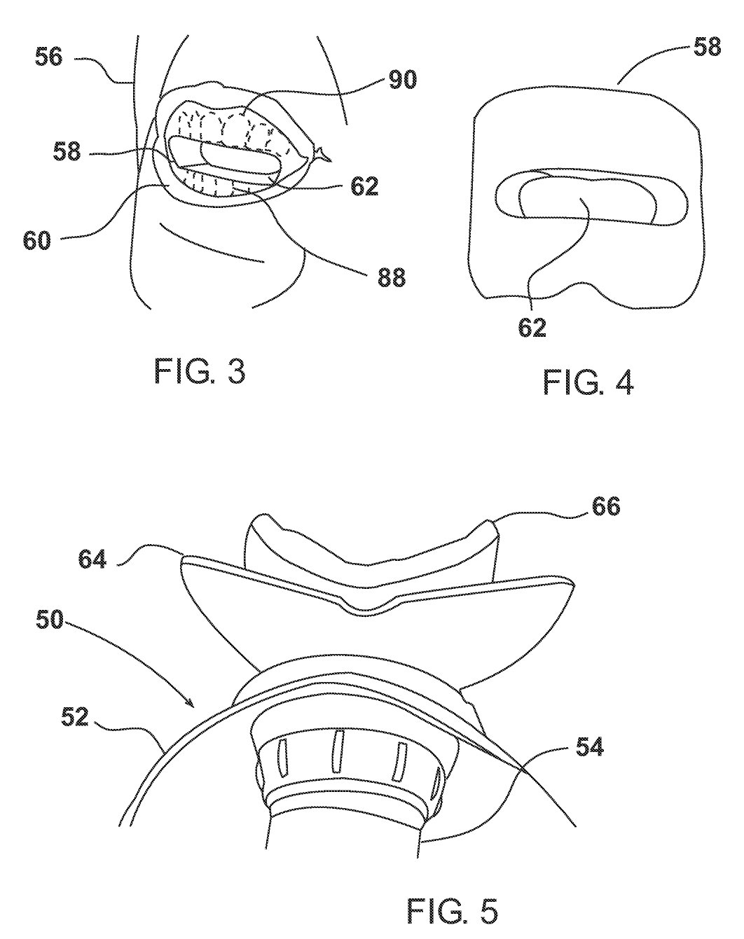

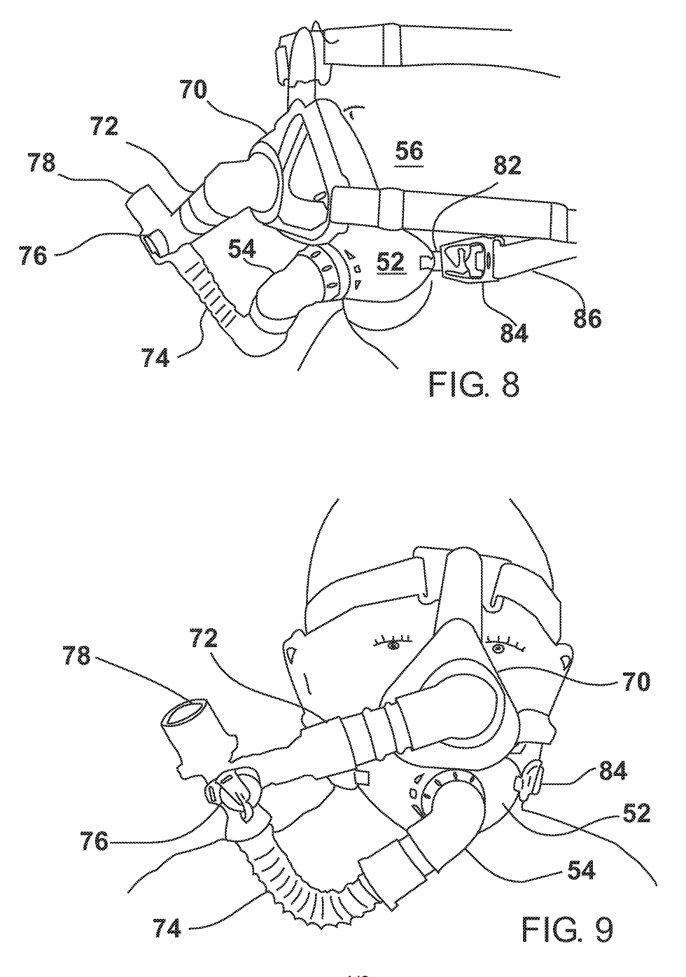

FIGS. 1 and 2 show an oral interface 50 attached to the mouth of a patient 56. An exterior flange 52 lies on the oral interface 50. The exterior flange 52 creates a seal with the lips 60 (FIG. 3) of the patient 56 when the exterior flange 52 is placed around the outside of the patient's mouth. The oral interface 50 may be connected to an oral interface fluid manifold 54. The fluid manifold may include plural fluid manifolds. The fluid manifold 54 may take the form of a tube. The oral interface fluid manifold 54 may be connected to an exterior source of air to provide breathable air to the patient 56. Connectors 82 on the exterior flange 52 may be attached to straps 86 (FIG. 8) to assist in holding the oral interface 50 on the patient's mouth. The fluid manifold is attached to the gas passage through which breathing gas can be provided through the gas passage to the patient.

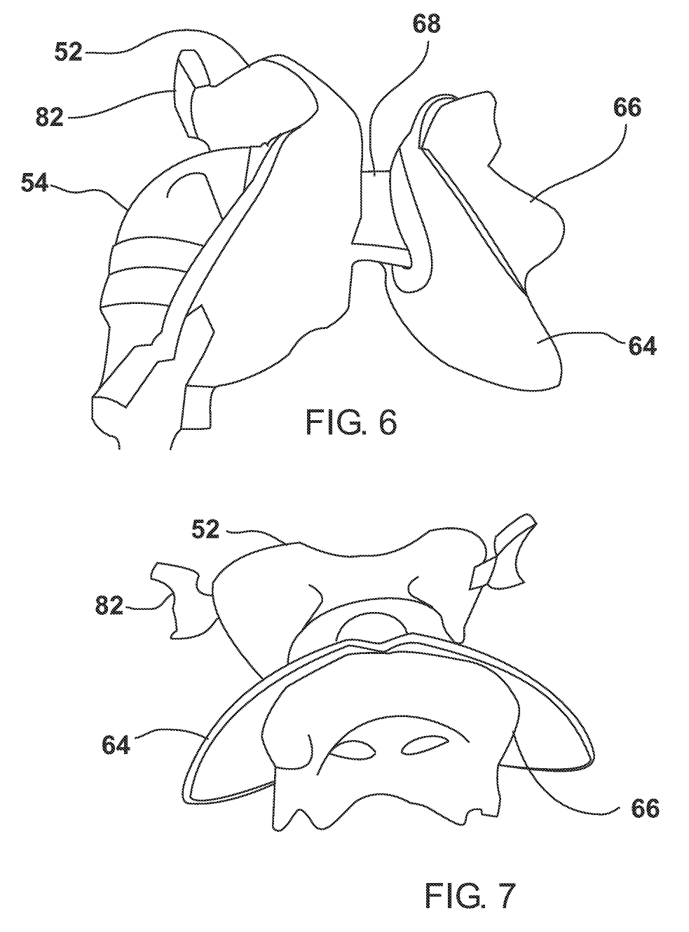

The lower portion may be connected, for example rigidly, to the upper portion of the mandibular positioning device. FIGS. 3 and 4 show this with a dental appliance 58 with a monolithic structure. The dental appliance 58 is placed over lower teeth 88 and upper teeth 90 of the patient 56. The dental appliance retains the lower teeth 88 and upper teeth 90. The dental appliance 58 functions as a mandible positioner and positions the mandible of the patient in relation to the maxilla of the patient. The dental appliance 58 may be made of a soft rubber. When the teeth of the patient are inserted into the soft rubber of the dental appliance 58 the teeth will be pulled into the position held by the soft rubber molding. For example, the dental appliance 58 may be molded so that the incisors of the molding are at the same level. If the patient's lower teeth and mandible are set back so that the incisors of the upper teeth lie in front of the incisors of the lower teeth, then when the patient places the dental appliance 58 into the patient's mouth, the patient's mandible will be protruded so that the incisors are at the same level. The dental appliance 58 lies within the lips 60 of the patient 56. There is an opening 62 on the anterior aspect of the dental appliance 58. The opening 62 may allow airflow into the patient's mouth while the dental appliance 58 is in use (FIG. 3). The dental appliance 58 may be attached to the oral interface 50 (FIG. 1) through a flexible insertion 68 (FIG. 6) which is inserted into the opening 62 of the dental appliance 58.

FIGS. 5-7 show a dental appliance 66 attached to the oral interface 50. The dental appliance 66 is a mandible positioner. The external flange 52 is shown pulled forward in FIGS. 5-7. The external flange 52 is shown in an operative position in FIGS. 1 and 2. An internal flange 64 provides sealing of the mouth from inside the lips 60 of the patient 56 (FIG. 3). The lips 60 of the patient 56 are sealed between the external flange 52 and the internal flange 64. The combination of two flanges allows sealing of the mouth at the level of the lips 60. The internal flange and external flange may be flexibly connected to each other. This way, the mouth seal does not require custom design. The dental appliance 66 may consist of a soft compliant material that fits between the incisors. The upper and lower arch appliances may be connected laterally by rubber straps which allow freedom of movement of the mandible laterally while the mandible is protruded moderately. The dental appliance 66 may position the mandible so that the incisors are at the same level; that is, the incisors are end to end. The dental appliance 66 is attached to the oral interface 50 by a flexible insertion 68 of the oral interface 50 through the upper and lower aspects of the dental appliance. The flexible insertion 68 allows freedom of adjustment and movement of the appliance relative to the teeth and to the upper lips. The flexible insertion 68 may be hollow to permit air to flow between the patient's mouth and the oral interface fluid manifold 54 without leaks. The flexible insertion 68 provides a connection between the oral interface 50 and the dental appliance 66 that is not rigid. The oral interface 50 may also be connected flexibly to the dental appliance 58 in a similar manner. The lack of a rigid connection between one of the dental appliances 58, 66 and the oral interface 50 allows the application of the oral interface 50 without custom design.

The dental appliance 66 serves to stabilize the mandible. The flexible insertion 68 connects the dental appliance 66 to the oral interface 50 in a flexible manner. The oral interface 50 is thereby constrained in its movement. The mandible is protruded and does not move posterially but it can move somewhat side-to-side depending upon the dental appliance that is used. The flexible connection of the oral interface fluid manifold 54 to the dental appliance allows the oral interface 50 to be adjusted to conform to the teeth, gums and lips of the patient 56 without having a custom made oral interface.

FIGS. 8 and 9 show the oral interface 50 in use with a nasal interface 70. The nasal interface 70 is a nasal mask in this embodiment. The oral interface fluid manifold 54 is connected by a flexible fluid manifold 74 to a valve 76. The flexible fluid manifold 74 may take the form of a tube. The nasal mask 70 is connected by a nasal interface fluid manifold 72 to the valve 76. The fluid manifold 72 may take the form of a tube. The valve 76 has an inlet 78 for providing breathable air into the system. A clip 84 attaches a strap 86 to the connector 82 on the external flange 52. The strap 86 holds the oral interface 50 to the patient's mouth. The oral appliance may be attached to the patient's mouth without the assistance of straps. The collection of the nasal mask 70, the oral mask 50, the nasal interface fluid manifold 72, the oral interface fluid manifold 54 and the valve 76 comprise a source of breathing gas for the patient 56. The inlet 78 may be attached to an exterior air source, such as for example, a low flow blower, an external CPAP device, a source of atmospheric air or any other source of breathable air. The oral interface 50 and the dental interface 66 function together as a breathing assistance apparatus in combination with a mandibular positioner.

The oral interface 50 and the nasal interface 70 can be both non-custom made and not arranged rigidly in relation to each other, thereby allowing the interface to be applicable to any face without custom design. The nasal interface has a nose seal, and the oral interface has a mouth seal.

The oral interface 50 and the nasal interface 70 may be used in a system providing continuous positive airway pressure (CPAP), controlled rebreathing or other type of breathing air to the patient. The oral interface 50 may be used without the nasal interface 70 and the nasal interface 70 may be used without the oral interface 50, although this may allow leaks through the nose or mouth if they are left uncovered. Additionally, the oral interface 50 may be used separately with a nasal occlusion device. The nasal interface 70 may be used as the only source of air to the patient by providing the flexible insertion 68 without an air passage. The oral interface 50 would then prevent any air from escaping the patient's mouth.

The connection between the oral interface 50 and the nasal interface 70 may be made through a fluid manifold which can vary in volume and in resistance. This allows the selection of an external dead space connection the oral interface 50 to the nasal interface 70. The selection of an external dead space can be useful in controlled rebreathing in treating patients with sleep apnea. In some embodiments, at least a portion of the fluid manifold is adjustable to vary the interior dead space volume. The flexible fluid manifold 74 as shown in FIGS. 8 and 9 between the oral interface 50 and the nasal interface 70 is flexible and easily adjustable by the patient.

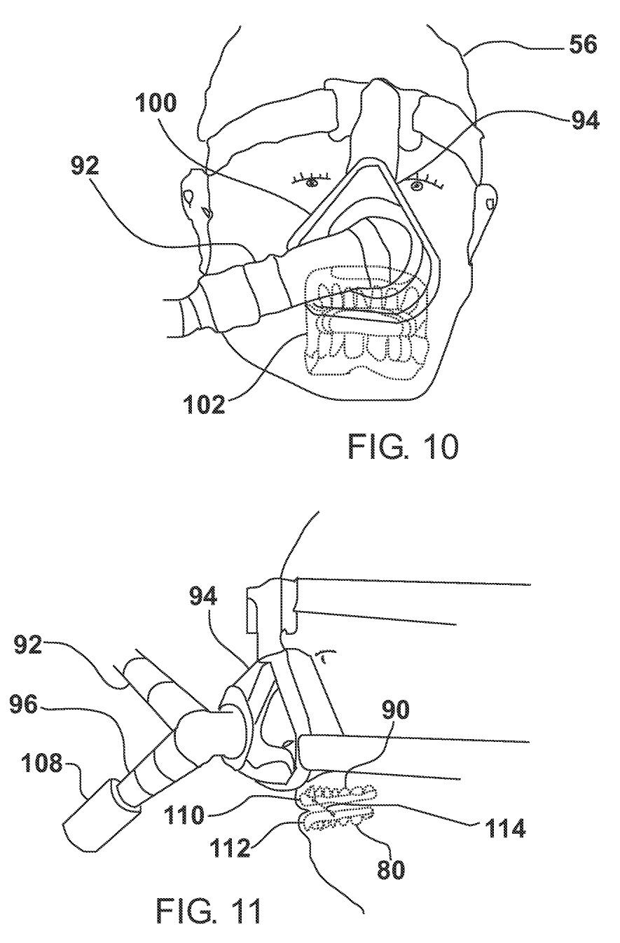

FIG. 10 shows an apparatus including a breathing assistance apparatus 100 and the mandible positioner 58 (FIG. 3) for positioning the mandible of the patient 56 with respect to the maxilla of the patient. The patient 56 has a breathing state. The breathing assistance apparatus 100 has a sensor, for example a flow meter 132 (FIG. 15), arranged to detect at least one element representative of the patient's breathing state. A source of breathing gas includes a patient interface 94 and has at least a first operable position and a second operable position. The source of breathing gas provides a different ratio of carbon dioxide and oxygen to the patient 56 when in the first operable position than in the second operable position. The source of breathing gas is movable between the first operable position and the second operable position in response to signals from the sensor. Thus, in the first operable position, the ratio of carbon dioxide to oxygen provided to the patient may be higher than the ratio of carbon dioxide to oxygen provided in the second operable position. The source of breathing gas includes a nasal mask 94 attached to a fluid manifold 92. The nasal mask 94 may be any type of nasal mask, for example, the nasal mask 94 may be the nasal interface 70 (FIG. 1).

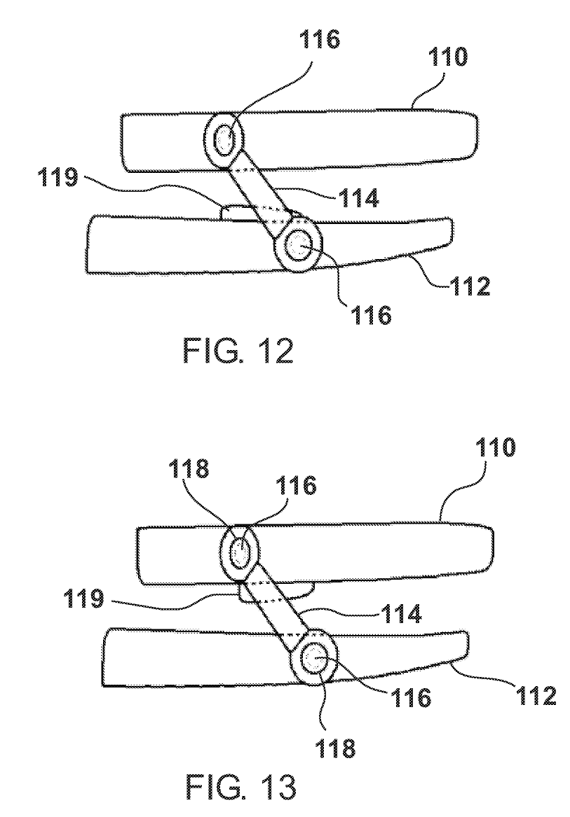

FIG. 11 shows the breathing assistance apparatus 100 of FIG. 10 with the mandibular protrusion device shown in FIG. 12. The source of breathing gas includes a valve 108 having at least a first valve position and a second valve position. The first and second operable positions of the source of breathing gas correspond to the valve 108 being in the first and second valve positions, respectively. The position of the valve 108 is varied in response to at least one detected element of the patient's breathing. The valve 108 is connected to an exit fluid manifold 96. The valve may be used to provide rebreathed air to the patient as described in the description corresponding to FIGS. 15-19. Re-breathed air, namely air that has recently passed through the lungs of a patient, has a higher ratio of carbon dioxide to oxygen than air in normal atmospheric conditions.

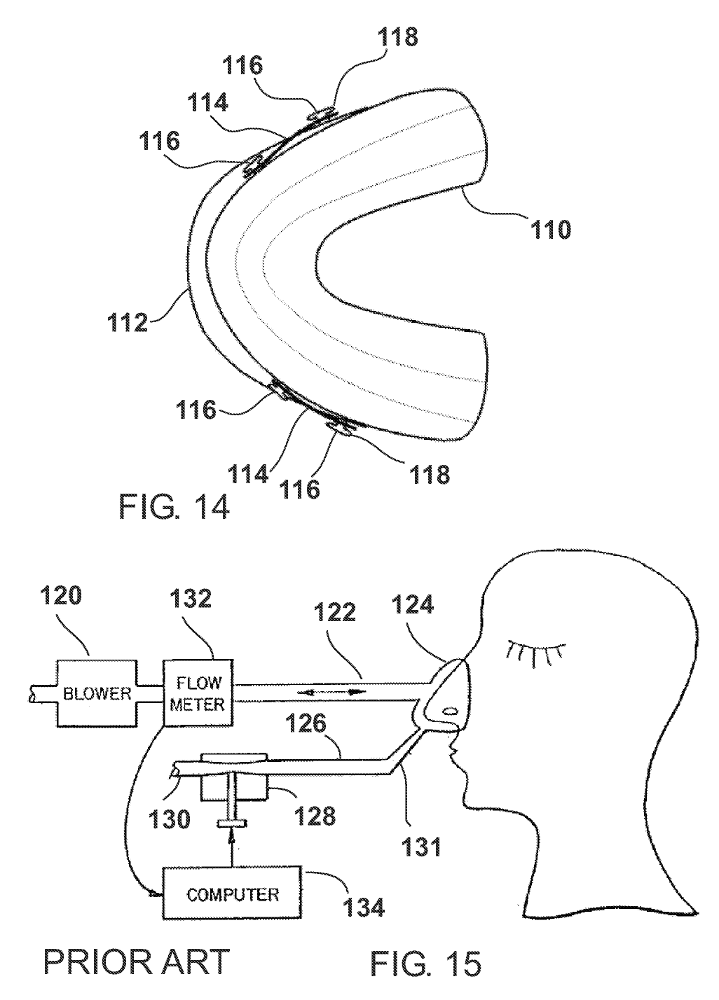

Referring to FIGS. 12-14, a mandibular protrusion device is formed from a full arch upper dental appliance 110 and lower dental appliance 112 connected by adjustable struts 114 which reposition the mandible ventrally and caudally. In FIGS. 12-14, the mandibular protrusion device is shown with the lower dental appliance 112 drawn forward into a therapeutic position in relation to the upper dental appliance 110. The protruding mechanism is situated lateral to the molars and allows graded protrusion of the mandible without encroaching on the tongue inside the dental arches. The struts 114 may be made of plastic and attach to the upper and lower dental appliances by fitting openings at the ends of the struts 114 over knobs 116 that protrude from the dental appliances 110, 112. The struts 114 should fit tightly on the knobs 116 to prevent the struts 114 from rotating on the knobs 116. To facilitate attachment of the struts 114 to the knobs 116, the heads of the knobs 116 may be made asymmetric, with a tab 118 extending outward one side, so that the openings in the struts 114 may first be fit over the shorter side of the head of the knob 116, then pressed over the tab 118. Other mechanisms may be used to hold the protrusion distance of the lower appliance 110 in relation to the upper appliance 112. A bite-opening wedge 119 may be glued to the occlusal surface of upper appliance (FIG. 13) or lower appliance (FIG. 12) near the most ventral molars. This forms an elevation extending 3-5 mm above the occlusal surface of the appliance and serves to open the bite enough to allow the tongue to extend ventrally between the incisors where it may be inserted into a tongue bulb (not shown).

The mandibular protrusion device enlarges the pharyngeal airway and makes it more difficult to close the airway. Enlarging the airway decreases the closing pressure inside the pharynx and the maximum open airway is enlarged. Thus, the pharynx does not narrow during breathing when the muscles are relaxed. Instead, the mandibular protrusion device holds the airway open and stabilizes the pharynx so that the pharynx does not flop around from open to close. Instability of the pharynx promotes central sleep apnea; hence use of the mandibular protrusion device decreases central sleep apnea.

If a patient fails to sufficiently respond to the mandibular protrusion device, controlled rebreathing may be used as well. Re-breathing, one manner of producing a source of breathing gas with a different ratio of carbon dioxide to oxygen than a patient would normally breath, may need to be controlled to avoid headaches and other problems caused by a continuous supply for excess CO₂. The amount of rebreathing can be adjusted. A sensor can be used to determine when re-breathing needs to be applied. For example, when a sensor determines that Cheyne Stokes breathing is occurring a small amount of rebreathing is provided during a period of increased breathing. The sensor may measure breath duration and the measurement may be converted to provide a breathing frequency. The sensor can detect Cheyne Stokes breathing when there is high tidal volume V and high breathing frequency F which results in high V×F. The small amount of rebreathing reduces loop gain when the gain is already too high. The amount of rebreathing can be adjusted. Breath duration is measured and converted to frequency. When breathing is normal, the patient simply breathes atmospheric air. It is preferable to use a system in which a low flow of atmospheric air is provided to a mask so that fresh air is always available. When Cheyne Stokes breathing is detected by a computer connected to the sensor, a valve is switched so that the patient goes from breathing a low flow of atmospheric air to a controlled amount of rebreathing for example from a dead space of air such as a fluid manifold connected to the mask. The increased CO₂ and decreased O₂ removes the effect of hyperventilation.

The mandibular protrusion device and controlled rebreathing may be applied in a non-CPAP setting. Mandibular protrusion with the use of an oral appliance opens stabilizes the pharyngeal airway during sleep, thereby eliminating in some cases the need for nasal CPAP. The dental appliance provides a usable and convenient attachment point for the nasal airway interface. The dental anchored nasal interface can be applied to produce a convenient and leak-free connection to the external dead space. This dental-anchored interface can either be a non-custom nose mask or full face mask such as is currently used in nasal CPAP therapy or it can be a custom nose mask or full face mask such as is currently used in nasal CPAP therapy or it can be a custom fitted oral/nasal interface. This interface allows the point of attachment for a circuit in which a valve controls the connection of the nasal airway either to the ambient atmosphere or to a rebreathing fluid manifold.

This interface-mounted valve is, in turn, controlled by a regulator which receives feedback from some measure of ventilation either through a sensor that measures volumetric movement of the chest or measures airflow at the mask. The regulator monitors tidal volume and frequency and calculates instantaneous ventilation and instantaneous alveolar ventilation. This allows identification of limit cycle behavior or near limit cycle behavior. If this behavior appears with the nasal interface connected to the atmosphere, the valve can be shifted to the rebreathing position wherein the subject rebreathes through the external dead space.

In summary, the combination of mandibular protrusion together with controlled rebreathing can be effectively accomplished with a leak-free interface anchored to the dental appliance. A binary valve, connecting the nasal airway either to the ambient atmosphere or to an external dead space is controlled by a regulator that receives feedback information regarding ongoing ventilation.

Possible embodiments of rebreathing apparatus are as follows. FIGS. 15-19 were originally described by Remmers et al. in U.S. Pat. No. 7,073,501, patented on Jul. 11, 2006.

FIG. 15 is a diagram illustrating the rebreathing apparatus of one active control embodiment. A source of breathing gas comprises a blower 120, a fluid manifold 122 and a patient interface 124. The fluid manifold 122 may take the form of a tube. Patient interface 124, comprising an oral interface and nasal occlusion device, produces an airtight tight seal to the face. The patient interface 124 may be used to provide continuous positive airway pressure (CPAP) treatment. A discussion of continuous positive airway pressure and a preferred continuous positive airway pressure apparatus is described in Remmers et al. in U.S. Pat. No. 5,645,053, “Auto-CPAP Systems and Method for Preventing Patient Disturbance Using Airflow Profile Information.” In conventional CPAP, a blower is used to maintain a relatively high constant pressure in a mask and to provide a bias flow of fresh air from the blower out the mask.

In FIG. 15 a fluid manifold 126 such as a tube is connected to an exhaust port 131 of the patient interface and conducts gas to the variable resistor 128. Alternatively, the valve can be located on the exhaust port 131 of the patient interface. Fluid manifold 122 is used as a dead space for rebreathing during some periods of the central sleep apnea respiration. When the valve 128 is open, no rebreathing occurs because all the exhaled gas is carried out fluid manifold 126 through valve 128 by the bias flow before inspiration occurs. When valve 128 is closed, the bias flow ceases and no expired air is conducted through fluid manifold 126. In this case, some partial rebreathing occurs because the expired air is conducted retrograde up fluid manifold 122 to the blower 120. The gases in the fluid manifold 126 have a higher concentration of carbon dioxide and a lower concentration of oxygen than room air. When the patient inspires, gas is conducted from the blower 120 to the patient and the previously expired gases are inhaled by the patient.

Normally, the bias flow of gas from the blower 120 through the patient interface 124 and out exit port 130 would be adequate to completely purge the system during the expiratory phase of the respiratory cycle so that no gas expired by the patient remains in the system. Thus, the gas inspired by the patient had a composition of atmospheric air (having generally O₂ concentration 21%; CO₂ concentration about 0%). Conversely, if the bias flow is reduced to zero by completely occluding exit port 130 with valve 128, the gas exhaled by the patient would fill the fluid manifold 122 connecting the patient interface 124 to the blower 120. Such expired gas would typically have a carbon dioxide concentration of 5% and an oxygen concentration of 16%. Upon inhalation, the patient would first inspire the high carbon dioxide, low oxygen mixture filling the fluid manifold, followed by inhalation of room air from the blower 120. Depending upon the length of the tubing this mixture could amount to rebreathing of 20 to 60 percent of the tidal volume. By varying the exhaust port outflow resistance, the degree of rebreathing between these limits can be varied and the inspired concentration of carbon dioxide and oxygen can be manipulated. A flow meter 132 connected to computer 134 is used to detect the flow of gases to and from the blower 120. The computer 134 is used to identify the periodicities in pulmonary ventilation caused by the central sleep apnea respiration and to control the valve 128 to cause rebreathing during certain periods of the central sleep apnea cycle.

The gas flow from the blower 120 comprises the bias flow (patient interface exit flow+leak flow) plus the respiratory airflow. The computer 134 monitors this flow and calculates the bias flow, leak flow, retrograde flow, retrograde expired volume and wash volume.

The computer 134 can detect the amplitude of the central sleep apnea cycle and to adjust the resistance of the valve 128 according. For example, if there are large variations in pulmonary ventilation during the central sleep apnea cycle, the valve 128 can be completely closed during the overbreathing period. If there are small variations in pulmonary ventilation during the central sleep apnea cycle, the valve 128 can be partially open during the overbreathing period. Thus, a higher level of rebreathing will occur when the variation in pulmonary ventilation during the central sleep apnea cycle is high than will occur when the variation in pulmonary ventilation during the central sleep apnea cycle is low.

Because of the low impedance of the CPAP blower 120, variations of the resistance in the outflow line cause very little change in patient interface pressure. Accordingly, the full range of variations in outflow resistance can be made without producing significant deviations in the desired CPAP patient interface pressure.

The flow meter 132 and computer 134 can quantitate the level of pulmonary ventilation. For example, the ratio of breath volume to breath period gives an indication of the level of the instantaneous pulmonary ventilation. Other indices such as mean or peak inspiratory flow rate could also be used.

A number of techniques are used to control the degree and timing of rebreathing with the valve 128 in order to eliminate central sleep apnea. One way of controlling rebreathing so as to reduce the central sleep apnea respiration is to anticipate the different cycles in the central sleep apnea respiration. For example, when the system anticipates a period of overbreathing, rebreathing is commenced by closing valve 128 as shown in FIG. 15. By the time overbreathing portion occurs, there is some level of rebreathing. Because of this, pulmonary gas exchange becomes less efficient during the period of overbreathing and, thereby, the resulting rise in lung oxygen and fall in lung carbon dioxide will be less. As a result, the level of oxygen in the blood does not get too high and the level of carbon dioxide does not get too low. This stabilizes the oxygen and carbon dioxide pressures in the arterial blood and thus will reduce the amplitude of subsequent underbreathing or the length of the apnea. When an underbreathing cycle is anticipated the system opens the valve 128 and rebreathing will no longer occur.

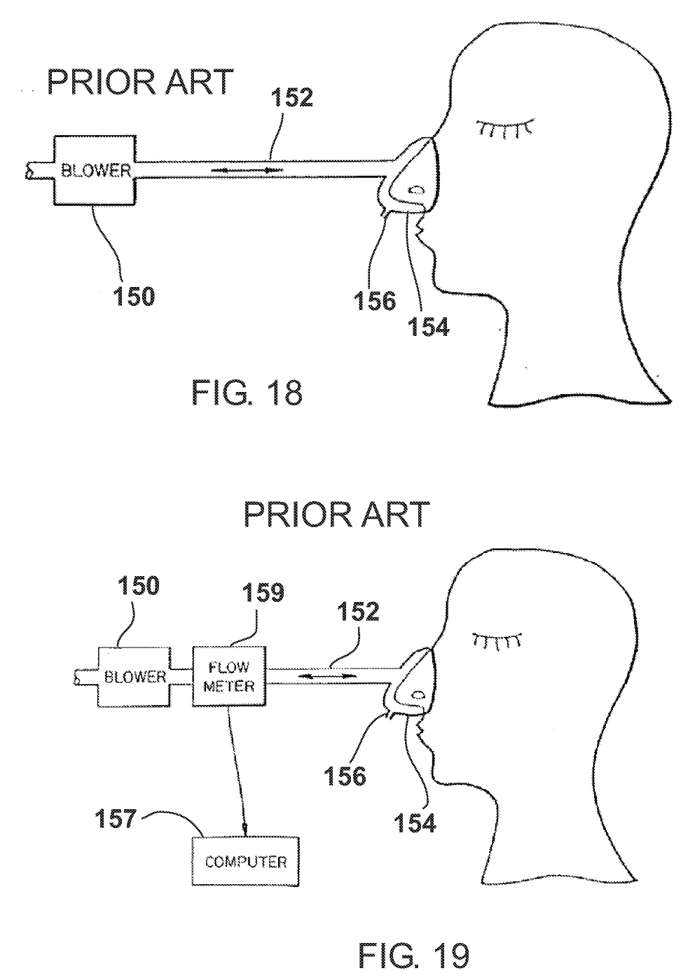

FIG. 18 is a diagram that illustrates a passive loop gain modulation system. FIG. 18 depicts a system using a gas-supply means such as the air blower 150 connected to a length of input tubing 152 and then to a patient interface 154. This system uses a simple fixed exit port for the patient interface 154. A tubing volume greater than that normally used with obstructive sleep apnea can be used with this system. For example, a ten-foot rather than six-foot tubing can be used. The blower 150 preferably has a very low impedance. That is, changes in the air flow do not significantly change the air pressure supplied by the blower. This can help maintain a relatively stable patient interface pressure even as the fluid manifold flow becomes retrograde.

Additionally, the air blower 150 is able to supply air pressure much lower than conventional CPAP blowers. The air blower 150 can be adjusted to supply pressures below 4 cm H₂O (preferably 2 cm H₂O or below). The ability to supply such small pressures allows for the retrograde flow as discussed below. The patient interface 154 is fitted about the patient's airway. During normal breathing, the air supplied from the blower 150 and fluid manifold 152 to the patient interface 154 does not cause any rebreathing because any exhaled air will be flushed before the next inhale period. During periods of heavy breathing, the preset gas flow pressure is set so that enough exhaled air flows retrograde into the fluid manifold such that during the next inhale period some expired gas is rebreathed. In this embodiment, the overbreathing occurs during certain periods of the sleep cycle associated with central sleep apnea. Rebreathing during periods of overbreathing during central sleep apnea tends to reduce the resulting spike in the blood oxygen level. Thus, the period of underbreathing following the overbreathing in the central sleep apnea sleep cycle will also be reduced.

The alternating periods of under- and overbreathing are reduced by the rebreathing which takes place during the periods of overbreathing. The rebreathing attenuates the arterial blood oxygen spike and the reduction in arterial P_{CO2} caused by the overbreathing. Thus, there is less underventilation when the blood reaches the chemoreceptors. Thus, the amplitude of the periodic breathing is reduced.

The embodiment of FIG. 18 is different than the conventional CPAP in that the preset gas flow pressure is lower and/or the patient interface exit hole is smaller than that used with conventional CPAP systems. By reducing the gas flow pressure from the typical CPAP gas flow pressures, and/or reducing the patient interface exit hole size, the retrograde flow during the overbreathing periods is produced.

The system of FIG. 18 has the advantage that it does not require active control of the blower pressure. The patient can be checked into a sleep center and the correct blower pressure and patient interface exit hole size set. Thereafter, the system can be placed on the patient's airway every night without requiring an expensive controller-based system. The preset blower gas pressure depends upon the air flow resistance caused by the exit 154, the normal exhale pressure and the overbreathing exhale pressure. If the gas-supply pressure system is an air blower 150, then by modifying the revolutions per minute of the air blower, the preset gas flow pressure can be set.

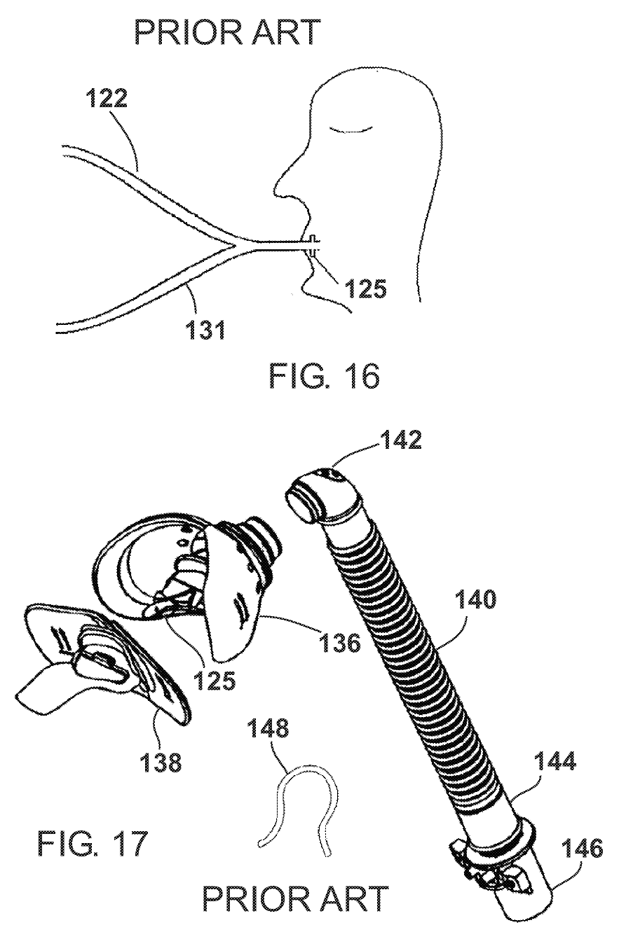

The air supply pressure for patients with central sleep apnea but without obstructive sleep apnea can be set at a relatively low level such as below 4 cm H₂O. The normal patient interface exit holes produce the desired effect at these pressures. The end-tidal F_{CO2} and inspired F_{CO2} can be monitored by a CO₂ meter with an aspiration line connected to the patient interface. Importantly, all mouth leaks should be eliminated by using a leak resistant patient interface in order to have expired gas move into the tubing 152. This can be achieved by applying a chin strap, or by using an oral appliance 125 (FIG. 16), or both. An alternative approach to difficult mouth leaks is to use a full face mask covering the mouth as well as the nose. This means that expired gas emanating from the nose or the mouth will travel retrogradely up the tubing 152 toward the blower. While it is important that leaks between the patient interface and the patient be minimized, it is also important that as much as possible of the exhaled air of the patient be conserved and made available for re-breathing. Hence, if the patient interface connects to the nose, then the mouth passageway should be blocked, and if the patient interface connects to the mouth, then the nasal passageway should be blocked. In either case, leaks through the unused passageway should be minimized. In some embodiments, the gas passage is through at least one of the nasal interface and the oral interface through which breathing gas can be provided to the patient. In some embodiments, the nasal interface and the oral interface each have a respective gas passage, and the fluid manifold is attached to the respective gas passages.

A simplified view of an oral appliance 125 is shown in FIG. 16. An example of an oral appliance 125 is illustrated in more detail in FIG. 17. The oral appliance 125 of FIG. 17 is fitted to a patient's mouth directly onto the lips, without using the teeth. The oral appliance 125 of FIG. 17 is held on a patient with a mask 136 that fits around a patient's airway and is secured with the use of straps and a pad 138 at the back of the patient's head. A fluid manifold 140, such as a tube, with normal bias ports 142 blocked, and low-flow bias flow port 144, connects to the CPAP apparatus through CPAP connection 146. The length of the fluid manifold 140 allows for a controlled amount of rebreathing.

A feature of the mode of action of the technology described in this patent document relates to the behaviour of the system during hyperventilatory periods. At these times, when such a hyperventilatory phase occurs, the patient generates a large tidal volume and short duration of expiration. Together, these induce rebreathing of expired gas that has flowed retrogradely into the CPAP conduit 140 connecting the CPAP blower to the patient interface such as oral appliance 125. For effective application of Low Flow CPAP an oral interface, such as the oral appliance 125, should be used in combination with nasal occlusion. Nasal occlusion may be obtained through plugs inserted in the nostrils or an external U-shaped clamp 148 (FIG. 17) similar to what would be used by a swimmer.

If the patient has an element of obstructive sleep apnea, the mandibular positioner may be used to progressively protrude the mandible of the patient until all evidence of upper airway obstruction is eliminated. Additionally, the patient interface pressure may be increased to assist in eliminating the upper airway obstruction. If the patient is receiving nasal CPAP as treatment for heart failure, patient interface pressure is set at the desired level (typically 8-10 cm H₂O). The bias flow (patient interface hole size) can then be reduced until central sleep apnea is eliminated without adding dead space.

In FIGS. 18 and 19, the flow through fluid manifold 152 depends upon the difference in pressure between the blower pressure (i.e., pressure at the outlet of the blower) and patient interface pressure. Blower pressure is set by the revolutions per minute (RPM) of the blower and will be virtually constant because the internal impedance of the blower is very low. When no respiratory airflow is occurring (i.e., at the end of expiration), patient interface pressure is less than blower pressure by an amount that is dictated by the flow resistive properties of the connecting fluid manifold and the rate of bias flow. This is typically 1-2 cm H₂0 pressure difference when bias flow is at 0.5-1.5 L/sec. When the patient interface is applied to the patient and the patient is breathing, patient interface pressure varies during the respiratory cycle depending upon the flow resistance properties of the connecting fluid manifold and the airflow generated by the patient. During inspiration the patient interface pressure drops, typically 1-2 cm H₂0, and during expiration the patient interface pressure may rise transiently a similar amount. During quiet breathing the peak-to-peak fluctuations in patient interface pressure are less than during heavy breathing or hyperpnea.

Thus, during quiet breathing the patient interface pressure rises during exhalation and this reduces the driving pressure difference between the blower and the patient interface, thereby reducing flow in the fluid manifold. If the expired tidal volume increases, however, peak expiratory flow will increase and this will be associated with a further increase in patient interface pressure. If patient interface pressure increases to equal blower pressure, flow in the fluid manifold will stop. When patient interface pressure exceeds blower pressure, flow in the fluid manifold will be in a retrograde direction, i.e., from the patient interface to the blower. Such retrograde airflow will first occur early in expiration and the volume of air which moves into the connecting fluid manifold will be washed out later in expiration as patient interface pressure declines and flow from the blower to the patient interface increases. However, if bias flow is low and the tidal volume is large, a large amount of retrograde flow will occur and a large volume of expired gas will move into the fluid manifold. Because the bias flow is small, the wash flow purging the fluid manifold will be small. In such a case, not all of the retrograde volume will be washed out before the next inspiration. As a consequence, the overall inspired gas will have a somewhat reduced oxygen concentration and an elevated carbon dioxide concentration.

There is little or no rebreathing during the normal breathing periods. The system of FIGS. 15-19 does not add dead space during the normal breathing periods. This is important because the addition of dead space can increase the concentration of carbon dioxide that is supplied to the bloodstream. It is assumed that if the increased carbon dioxide level persists for multiple days, the body will readjust the internal feedback system an undesirable manner.

FIG. 19 shows the device of FIG. 18 with the addition of a computer 157 and flow meter 159. The flow meter 159 is used to detect the desired air flow in the fluid manifold 152. The blower 150 can then be adjusted so that there is retrograde flow during periods of overbreathing and no retrograde flow otherwise. The device of FIG. 19 can be used to calibrate the device of FIG. 18 for an individual patient.

The mandibular positioners shown in the drawings may be substituted with any mandibular protrusion device that can adjust the position of the mandible with respect to the maxilla. An example of a mandibular protrusion device is one that has a full arch and tooth connection and is preferably custom fitted. That is, a mold is made of the jaw and then a perfectly fitting device can be made. The mandible and the maxilla are connected by the mandibular protrusion device so that a forward force may be placed on the mandible. The mandibular protrusion device is preferably adjustable so that the forward force on the mandible can be progressively increased. The mandibular protrusion device may be worn by a patient through the night.

Controlled rebreathing may also be used in conjunction with CPAP. CPAP is a well-established therapy for breathing instability. In CPAP a patient is provided with a controlled over pressure of air through a mask connected to a blower. Note that continuous breathing is disclosed for use in combination with CPAP. The use of a low flow of air is a form of flow CPAP, enough to ventilate the mask.

Another option is to use mandibular positioning with a low flow of oxygen. The low flow of oxygen is a source of controlled air flow. The interface may provide atmospheric air or rebreathed air to the patient when the source of breathing gas is in the first position. The source of breathing gas could then provide the interface with a controlled supply of oxygen from the source of controlled air flow in the second operable position. The effect of the supply of a low flow of oxygen is to reduce the loop gain in the chemoreflex control loops of the patient. The low flow of oxygen may be provided at a rate of several litres per minute supplied through nasal prongs or a loose fitting mask.

CPAP may also be provided to the patient through either the nasal interface, the oral interface or through both the oral and nasal interfaces. The application of CPAP may be provided in conjunction with mandibular protrusion. A supply of external carbon dioxide may be provided rather than providing rebreathed air to the patient in order to provide different levels of carbon dioxide and oxygen to the patient.

The external dead space may be any confined space of any shape as long as it retains a volume of exhaled air. The fluid manifold may be any material that defines a flow passage for transfer of fluids, mostly gases, when fluid transfer is required, or holding of fluids, mostly gases, when fluid holding is required. In many instances, a tube will suffice for the fluid manifold, but the manifold may have an arbitrary shape.

In some embodiments the apparatus comprises one or more of the following sets of features: a nasal interface having a nose seal, an oral interface having a mouth seal, and a gas passage through at least one of the nasal interface and the oral interface through which breathing gas can be provided to the patient; and an oral interface having a mouth seal comprising an internal flange for sealing around an interior of the mouth, and an external flange for sealing around an exterior of the mouth. When the first and second sets of features are both present, the first oral interface and the second oral interface are understood to be the same interface.

Immaterial modifications may be made to the embodiments described here without departing from what is covered by the claims.

What is claimed is:

1. An apparatus, comprising:
a mandible positioner for positioning the mandible of a patient with respect to the maxilla of the patient, the patient having a breathing state; and
a breathing assistance apparatus comprising:
a sensor arranged to detect at least one element representative of the patient's breathing state;
a source of breathing gas including a patient interface and having at least a first operable position and a second operable position, the source of breathing gas being configured to provide a different ratio of carbon dioxide and oxygen to the patient when in the first operable position than in the second operable position; and
the source of breathing gas being movable between the first operable position and the second operable position in response to signals from the sensor.

2. The apparatus of claim 1 in which:
the source of breathing gas further comprises a valve, the valve having at least a first valve position and a second valve position; and
the first and second operable positions of the source of breathing gas corresponding to the valve being in the first and second valve positions.

3. The apparatus of claim 2 in which the source of breathing gas is configured to provide atmospheric air to the patient when the valve is in the second valve position.

4. The apparatus of claim 3 further comprising one or more of the following features:
the source of breathing gas is configured to provide breathing gas to the patient with an increased concentration of carbon dioxide gas and reduced concentration of oxygen gas relative to atmospheric air when the valve is in the first valve position;
the source of breathing gas is configured to change from the second operable position to the first operable position when the element representative of the patient's breathing state is determined to represent an abnormal breathing state
the mandible positioner comprises an upper portion constructed to attach at least partially to the maxilla of a patient, a lower portion constructed to attach at least partially to the mandible of a patient and an elastic to move the lower portion relative to the upper portion; and
the interface comprises a nasal mask.

5. The apparatus of claim 1 in which the source of breathing gas further comprises a fluid manifold operably connected to the interface and the interface further comprising an exit.

6. The apparatus of claim 5 in which in the first operable position of the source of breathing gas some exhaled gases from the patient flow retrograde into the fluid manifold and away from the exit so that rebreathing occurs when the next inhale portion of the patient's breathing occurs.

7. The apparatus of claim 1 in which the apparatus further comprises a source of controlled gas flow and when the source of breathing gas is in the second operable position, the source of controlled gas flow provides the interface with a controlled supply of gas flow with a higher concentration of oxygen than atmospheric air.

8. The apparatus of claim 7 in which the interface provides atmospheric air to the patient when the source of breathing gas is in the first operable position.

9. A method for assisting the breathing of a patient, comprising the steps of:
protruding the mandible of a patient with a mandibular protrusion device;
detecting the breathing of a patient;
determining whether abnormal breathing conditions are present; and
changing an amount of carbon dioxide concentration provided to the patient when abnormal breathing conditions are determined to be present.

10. The method of claim 9 in which changing the amount of carbon dioxide concentration provided to the patient further comprises one or more of:
increasing the amount of carbon dioxide concentration provided to the patient; and
connecting at least one breathing orifice of the patient to an external dead space.

11. The method of claim 9 in which detecting the breathing of a patient further comprises:
operably connecting a sensor to at least one breathing orifice of the patient; and
measuring the flow of gas from the at least one breathing orifice of the patient using the sensor.

12. An apparatus for assisting the breathing of a patient having a breathing state, the apparatus comprising:
a mandibular positioning device;
an interface adapted to provide breathing gas to a breathing orifice of the patient;
a sensor attached to the interface, the sensor detecting at least one element representative of the patient's breathing state;
a fluid manifold connected to an exterior gas source, the fluid manifold connected to the interface;
an exit connected to the interface; and
a valve operably connected to the sensor to vary the amount of flow of exhaled gases from the patient into the fluid manifold.

13. The apparatus of claim 12 in which when the at least one element representative of the patient's breathing states is determined to be abnormal the valve is adjusted in response to signals from the sensor so that some exhaled gases from the patient flow retrograde into the fluid manifold towards the exterior gas source and away from the exit.

14. An apparatus for assisting the breathing of a patient having a breathing state, the apparatus comprising:
a mandibular positioning device;
an interface adapted to provide breathing gas to a breathing orifice of the patient;
a fluid manifold connected to the interface;
a sensor attached to the fluid manifold, the sensor detecting at least one element representative of the patient's breathing state; and
an exterior pressurized gas source connected to the interface and providing gas with a higher content of oxygen gas than atmospheric air to the patient in response to signals from the sensor.

15. An apparatus for interfacing with a patient to allow breathing gas to be provided to the patient, the apparatus comprising:
a mandibular positioning device for positioning the mandible of a patient with respect to a maxilla of the patient; the apparatus further comprising feature A or feature B or both feature A and feature B:
where feature A comprises a nasal interface having a nose seal, an oral interface having a mouth seal, and a gas passage through at least one of the nasal interface and the oral interface through which breathing gas can be provided to the patient; and
where feature B comprises an oral interface having a mouth seal comprising an internal flange for sealing around an interior of the mouth, and an external flange for sealing around an exterior of the mouth.

16. The apparatus of claim 15, in which the apparatus comprises feature A, and further comprising a fluid manifold attached to the gas passage through which breathing gas can be provided through the gas passage to the patient.

17. The apparatus of claim 16 in which the nasal interface and the oral interface each have a respective gas passage, and in which the fluid manifold is attached to the respective gas passages for the provision of breathing gas to the patient through the nasal interface and the oral interface.

18. The apparatus of claim 16, further comprising one or more of the following:
the fluid manifold comprises a flexible connection between the nasal interface and the oral interface; and
at least a portion of the fluid manifold is adjustable to vary an interior dead space volume.

19. The apparatus of claim 15 in which the apparatus comprises feature B and the internal flange and external flange are flexibly connected to each other.

20. The apparatus of claim 15 in which the mandibular positioning device is connected to the oral interface through a flexible insertion.

21. The apparatus of claim 15 in which the mandibular positioning device comprises:
an upper portion constructed to attach at least partially to the maxilla of a patient;
a lower portion constructed to attach at least partially to the mandible of a patient, the lower portion being connected to the upper portion; and further comprising one of the following features:
the lower portion is movable laterally relative to the upper portion; and
the upper portion and the lower portion are rigidly connected.
